(19) United States
(12) Patent Application Publication
(10) Pub. No.: US 2026/0120846 A1
(43) Pub. Date: Apr. 30, 2026

(54) OUTLIER DETECTION IN IMAGE GROUPS

(71) Applicant: Microsoft Technology Licensing, LLC, Redmond, WA (US)

(72) Inventors: Luke Morse SHIPMAN, Newburyport, MA (US); Jameson MERKOW, Seattle, WA (US); Ivan TARAPOV, Redmond, WA (US)

(21) Appl. No.: 18/931,018

(22) Filed: Oct. 29, 2024

(51) Int. Cl. G16H 30/40; G06V 10/44; G06V 10/75; G16H 30/20

(52) U.S. Cl. CPC G16H 30/40; G06V 10/44; G06V 10/758; G16H 30/20

(57) ABSTRACT

A computerized method determines whether an image group is an outlier with respect to a set of reference image groups. Reference feature vectors are generated for each image in the reference image groups and, using those reference feature vectors, reference statistical vectors are generated. Each reference statistical vector is associated with a reference image group. An input image group is received, and input feature vectors are generated based on the images of the input image group. The input feature vectors are used to generate an input statistical vector associated with the input image group. Outlier analysis is performed using the input statistical vector and the reference statistical vectors and it is determined that the input image group is an outlier with respect to the reference image groups based on the performed outlier analysis. An automatic data analysis operation is then performed based on the outlier status of input image group.

### BACKGROUND

Imaging analysis is a complex process that is important for medical research and other related fields. To perform such analysis, data sets of pluralities of image groups (e.g., groups of images associated with a single scan session or study) must be sorted and/or filtered with respect to categories. Machine learning models can be trained to automatically do such sorting and filtering, but training models for specific image group categories can become expensive and time-consuming.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

A computerized method for determining whether an image group is an outlier with respect to a set of reference image groups is described. Reference feature vectors are generated for each image in the reference image groups and, using those reference feature vectors, reference statistical vectors are generated. Each reference statistical vector is associated with a reference image group. An input image group is received, and input feature vectors are generated based on the images of the input image group. The input feature vectors are used to generate an input statistical vector associated with the input image group. Outlier analysis is performed using the input statistical vector and the reference statistical vectors and it is determined that the input image group is an outlier with respect to the reference image groups based on the performed outlier analysis. A data analysis action is then caused to be performed on an image group from which the input image group is excluded based on the outlier status of the input image group.

### BRIEF DESCRIPTION OF THE DRAWINGS

The present description will be better understood from the following detailed description read considering the accompanying drawings, wherein:

FIG. 1 is a block diagram illustrating an example system configured for generating reference statistical vectors from reference image groups and determining whether an input image group is an outlier with respect to the reference image groups;

FIG. 2 is a flowchart illustrating an example method for determining whether an input image group is an outlier with respect to a plurality of reference image groups;

FIG. 3 is a flowchart illustrating an example method for categorizing an input image group using a plurality of reference image group categories;

FIG. 4 is a flowchart illustrating an example method for filtering outlier image groups from a training data set of image groups;

FIG. 5 is a flowchart illustrating an example method for determining whether an input medical imaging study is an outlier with respect to a plurality of reference medical imaging studies;

FIG. 6 is a diagram illustrating structures of two example medical imaging studies; and

FIG. 7 illustrates an example computing apparatus as a functional block diagram.

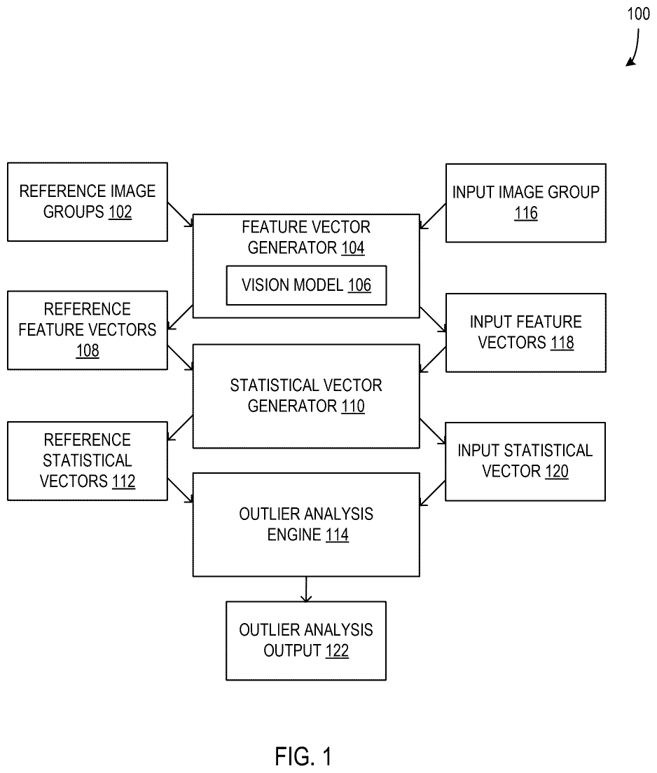

Corresponding reference characters indicate corresponding parts throughout the drawings. In FIGS. 1 to 7, the systems are illustrated as schematic drawings. The drawings may not be to scale. Any of the figures may be combined into a single example or embodiment.

### DETAILED DESCRIPTION

Aspects of the disclosure provide systems and methods that identify outlier image groups from input image groups with respect to reference image groups and enable the categorization and/or filtering of the input image groups as described herein. The reference image groups are analyzed by generating reference statistical vectors that reflect statistical data values of each reference image group. The method for generating the statistical vectors includes generating a feature vector for each image in the reference image groups and then statistically analyzing the feature vectors of each reference image group to generate a statistical vector for each reference image group. Similarly, when determining whether an input image group is an outlier with respect to some or all of the reference image groups, an input statistical vector is generated for the input image group and outlier analysis is performed using the input statistical vector and the reference statistical vectors. Based on the outlier analysis, it is determined whether the input image group is an outlier with respect to the reference image groups.

Aspects of the disclosure operate in an unconventional manner at least by generating a statistical vector for each image group that is being analyzed. The statistical vectors are generated in such a way (e.g., generating reference feature vectors of a plurality of images in reference image groups and generating reference statistical vectors for the reference image groups using the generated reference feature vectors of images included in the reference image groups) that the statistical vectors have a normalized size, regardless of the contents of each image group. As a result, an image group with multiple images (e.g., 200) can be efficiently compared (e.g., in a technical computing sense, such as via efficient use of computing resources) to image groups with different number of images (e.g., 50) by performing outlier analysis on the associated statistical vectors (e.g., performing outlier analysis using the input statistical vector and the reference statistical vectors). In some examples, the disclosure enables image groups to be automatically sorted, filtered, and/or otherwise categorized without performing expensive custom training of machine learning models. Thus, the described two-stage generation of statistical vectors and outlier analysis thereof reduces the use of computing resources (e.g., processing, memory, and/or bandwidth) and reduces the time required by computing systems.

FIG. 1 is a block diagram illustrating an exemplary system 100 configured for generating reference statistical vectors 112 from reference image groups 102 and determining whether an input image group 116 is an outlier with respect to the reference image groups 102. In some examples, the reference image groups are analyzed by a feature vector generator 104 and/or an associated vision model 106 to generate reference feature vectors 108 or other embeddings. The reference feature vectors 108 are analyzed to determine statistical values associated therewith and those statistical values are used to generate reference statistical vectors 112 using a statistical vector generator 110. The reference statistical vectors 112 are stored for later use in comparison with input data.

In some examples, an input image group 116 is analyzed using the feature vector generator 104 and/or the associated vision model 106 to generate input feature vectors 118 (or other embeddings) and those input feature vectors 118 are analyzed by statistical vector generator 110 to determine statistical values associated therewith. The statistical vector generator 110 generates an input statistical vector 120 associated with the input image group 116 using the statistical values associated with the input feature vectors 118. To determine whether the input image group 116 is an outlier with respect to the reference image groups 102, the input statistical vector 120 and the reference statistical vectors 112 are provided as input to the outlier analysis engine 114. The outlier analysis engine 114 performs outlier algorithms and/or processes to generate outlier analysis output 122 that indicates whether the input image group 116 is an outlier with respect to one or more of the reference image groups 102.

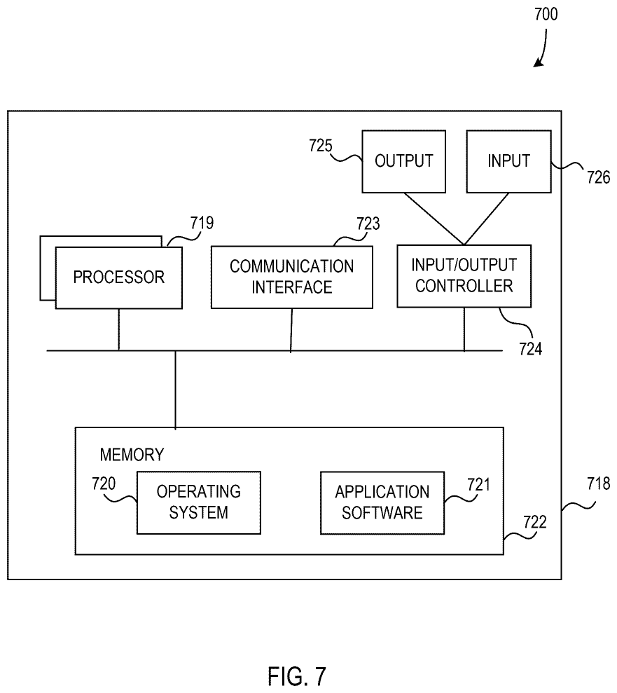

Further, in some examples, the system 100 includes one or more computing devices (e.g., the computing apparatus of FIG. 7) that are configured to communicate with each other via one or more communication networks (e.g., an intranet, the Internet, a cellular network, other wireless network, other wired network, or the like). In some examples, entities of the system 100 (e.g., feature vector generator 104, vision model 106, statistical vector generator 110, and/or outlier analysis engine 114) are configured to be distributed between the multiple computing devices and to communicate with each other via network connections. For example, the feature vector generator 104 is executed on a first computing device and the statistical vector generator 110 is executed on a second computing device within the system 100. The first computing device and second computing device are configured to communicate with each other via network connections. Alternatively, in some examples, other components of the feature vector generator 104 (e.g., the vision model 106 and/or interfaces exposed for receiving image groups, etc.) are executed on separate computing devices and those separate computing devices are configured to communicate with each other via network connections during the operation of the feature vector generator 104. In other examples, other organizations of computing devices are used to implement system 100 without departing from the description.

In some examples, the reference image groups 102, or exemplar image groups, include a plurality of subgroups of images, wherein images in the subgroups are associated with each other. For instance, in an example, the reference image groups 102 include groups of images associated with medical imaging procedures, such as computed tomography (CT) scans, magnetic resonance imaging (MRI) scans, and/or other medical imaging scans that include a plurality of cross-sectional images, or series of images, of a patient that can provide a three-dimensional representation of aspects of the patient's body when viewed together (e.g., a collection of 10-1000 images in a series, wherein each image represents a slice of the body imaged). In such an example, a single reference image subgroup is associated with a single CT scan series of a patient, wherein the reference image subgroup includes the series of images captured during the CT scan. In some cases, multiple CT scan series are combined to form a study for a patient, wherein each series of a study is used to image a different parameter, such as different phases of breathing, different phases of contrast liquid washout, different scanner parameters, or the like. The reference image groups 102 then include a plurality of such subgroups representing a plurality of CT scan series of patients. In some cases, the reference image groups 102 include a plurality of CT scans that are of the same category (e.g., CT scans that are acquired during a single patient visit, also known as a study). Alternatively, in other cases, the reference image groups 102 include a plurality of CT scans that includes scans of different categories. The reference image subgroups in such reference image groups 102 are tagged with or otherwise associated with a category indicator that can be used by the system 100 as described herein.

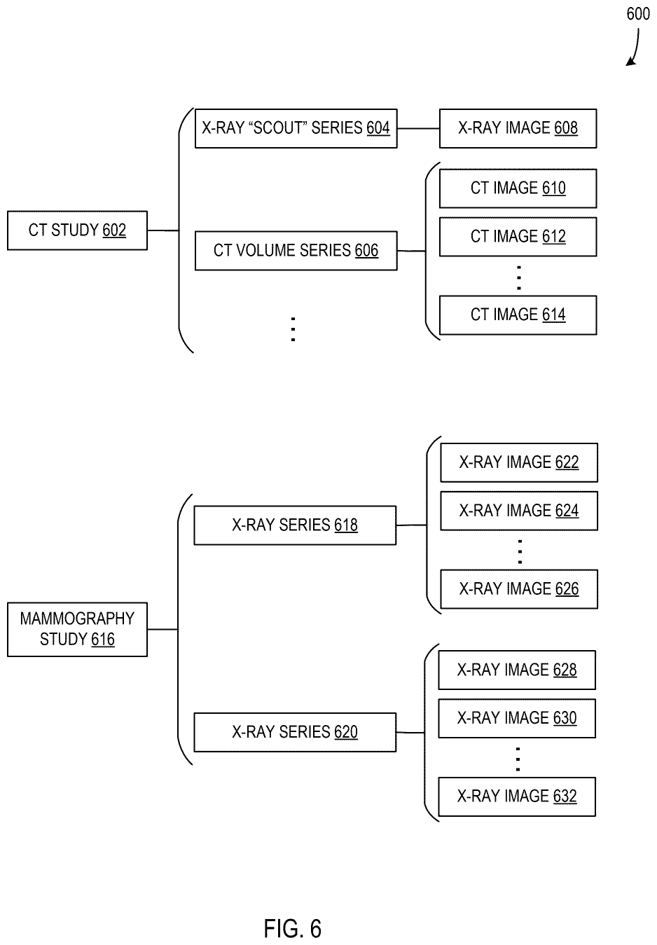

In some examples, the reference image groups 102 are medical imaging studies (e.g., the studies of FIG. 6). The medical imaging studies include one or more medical imaging series, which include one or more medical images. A medical imaging series includes images associated with a type of scan or imaging process, such as X-rays, MRI, CT scans, Ultrasound, positron emission tomography (PET) scans, or the like. While each medical imaging series includes images from a single type of scan or imaging process, a medical imaging study can include medical imaging series from multiple types of scans or imaging processes as well as other types of data. For instance, in an example, a medical imaging study of a patient includes a first medical imaging series from an X-ray, a second medical imaging series from a CT scan, and a third medical imaging series from an Ultrasound. In other examples, more, fewer, or different types of medical imaging series are included in the medical imaging studies without departing from the description. It should be understood that a first medical imaging study may have very different series with different quantities of images than a second medical imaging study and the described systems and methods can be used to compare the first and second medical imaging studies as described herein. Further, the systems and methods can be used to compare medical image series to each other as well.

While many of the examples herein are directed to medical imaging, in other examples, other types of images are used without departing from the description.

The feature vector generator 104 includes hardware, firmware, and/or software configured to receive the image data of reference image groups 102 and to generate reference feature vectors 108 from that received image data. In some examples, a vision model 106 is used in the generation of the reference feature vectors 108. Further, in some examples, the feature vector generator 104 analyzes the image data of each image in a reference image group 102 or a subgroup thereof. Using the analysis, a reference feature vector 108 is generated for each image in the reference image group 102 or subgroup being analyzed. Each reference feature vector 108 includes a plurality of data values that represent features that are present or not present in the images, and/or the degree to which features are present in the images (e.g., for an image feature, a first image includes an obvious instance of the image feature and therefore has a high data value (e.g., more than a threshold such as .95 on a scale from zero to one) in an associated reference feature vector entry for that image feature while a second image includes only a partial instance of the image feature and therefore has a relatively lower data value (e.g., less than a threshold such as .5 on a scale from zero to one) in an associated reference feature vector entry for that image feature).

In some such examples, the vision model 106 is trained using machine learning (ML) techniques to generate reference feature vectors 108 and, during analysis of the reference image groups 102, the vision model 106 applies its trained operations to the images of the reference image groups 102 to generate the reference feature vectors 108 as described herein. In other examples, more or different types of ML models are used without departing from the description. Alternatively, in still other examples, algorithms or other processes that do not include ML models are used to generate the reference feature vectors 108 without departing from the description.

Further, in some cases, the feature vector generator 104 is configured to generate the reference feature vectors 108 using encoding processes and/or by transforming the image data to be represented in a lower-dimensional embedding space.

The statistical vector generator 110 includes hardware, firmware, and/or software configured to receive reference feature vectors 108 as input and to generate reference statistical vectors 112 as output. In some examples, the statistical vector generator 110 receives a group of reference feature vectors 108 that are associated with a reference image subgroup of a category. The statistical vector generator 110 calculates or otherwise determines statistical values about the group of reference feature vectors 108, such as average number of data entries in the reference feature vectors 108, average values of specific data entries in the reference feature vectors 108, standard deviation associated with data values in the reference feature vectors 108, minimums and/or maximums of values of data entries in the reference feature vectors 108, median values and/or associated percentile values (e.g., the 25% value, the 75% value) of data entries in the reference feature vectors 108, or the like.

In some examples, each reference statistical vector 112 includes the same quantity of value entries associated with the same statistical value types, such that reference statistical vectors 112 can efficiently be compared to each other and/or to input statistical vectors 120 as described herein. For instance, in an example, each reference statistical vector 112 includes a first data entry that represents a count value of the quantity of images in the associated reference image group 102. Thus, the image count of each reference image group 102 can be compared by comparing the first data entries in the associated reference statistical vectors 112.

The outlier analysis engine 114 includes hardware, firmware, and/or software configured to perform outlier analysis (e.g., an outlier algorithm or other outlier evaluation process) on statistical vectors 112 and 120 that are provided as input. The outlier analysis engine 114 generates outlier analysis output 122 that includes an indication as to whether an input statistical vector 120 is an “outlier” with respect to the reference statistical vectors 112 that are input to the outlier analysis engine 114. In some examples, the outlier analysis engine 114 performs outlier detection methods or algorithms such as Z-Score, Local Outlier Factor (LOF), Isolation Forest, DBSCAN, and/or Coresets methods. Alternatively, or additionally, in other examples, more and/or different outlier detection methods, algorithms, or techniques are used by the outlier analysis engine 114 without departing from the description.

In some examples, the outlier analysis engine 114 determines whether the statistical data values of the input statistical vector 120 differ significantly from the corresponding statistical data values of the reference statistical vectors 112. The generated outlier analysis output 122 includes an indicator (e.g., a binary bit equaling 1 or 0) that indicates whether the input statistical vector 120 is an outlier, or significantly different, with respect to the reference statistical vectors 112. Further, in some such examples, the outlier analysis engine 114 calculates or otherwise determines a difference value that indicates a degree to which the input statistical vector 120 differs from the reference statistical vectors 112. The difference value is compared to a defined difference threshold (e.g., a value) and, if the difference value exceeds the difference threshold, the input statistical vector 120 is considered an outlier with respect to the reference statistical vectors 112. The outlier detection method used by the outlier analysis engine 114 is used to determine the difference value in such examples.

It should be understood that, in some examples, after the generation of the reference statistical vectors 112, the system 100 enables an input image group 116 to be provided, resulting in the generation of input feature vectors 118 of the images in the input image group 116 using the feature vector generator 104 as described herein. Then, the input feature vectors 118 are used by the statistical vector generator 110 to generate a single input statistical vector 120 associated with the input image group 116. Thus, the input statistical vector 120 can be compared to one or more of the reference statistical vectors 112 to determine whether the input image group 116 is an outlier or inlier with respect to the reference image groups 102 and/or one or more reference image subgroups within the reference image group 102.

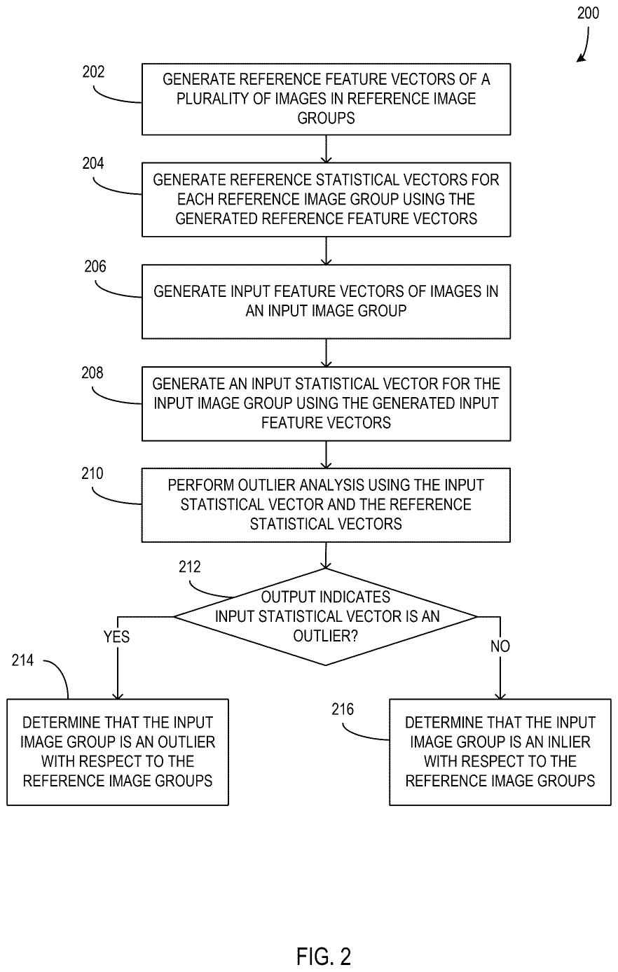

It should be understood that, in other examples, the system 100 includes, in isolation or combination, other features and/or aspects described herein with respect to FIGS. 2, 3, 4, and/or 5 without departing from description.

FIG. 2 is a flowchart illustrating an exemplary method 200 for determining whether an input image group is an outlier with respect to a plurality of reference image groups. In some examples, the method 200 is executed or otherwise performed in a system such as system 100 of FIG. 1.

At 202, reference feature vectors (e.g., reference feature vectors 108) of a plurality of images in reference image groups are generated. In some examples, the reference feature vectors are generated using a feature vector generator 104 as described above. One reference feature vector is generated for each image, such that a reference image group that contains one hundred images is associated with one hundred of the generated reference feature vectors. Further, in some examples, a vision model 106 is used to generate the reference feature vectors such that each reference feature vector is of the same size (e.g., the same quantity of data values within the vector). It should be understood that, in some examples, the vision model is trained in a general way and not trained specifically for use with the particular reference image groups.

At 204, reference statistical vectors (e.g., reference statistical vectors 112) for each reference image group are generated using the reference feature vectors. That is, a single reference statistical vector is generated for a reference image group that includes one hundred images, wherein the reference statistical vector is generated using the one hundred reference feature vectors associated with the reference image group. In some examples, the generated reference statistical vectors include statistical data values obtained from analyzing the reference feature vectors of the reference image group as described herein. Further, in some examples, the reference statistical vectors are generated using a statistical vector generator 110 as described above with respect to FIG. 1.

In some examples, generating the reference statistical vector for a reference image group of the reference image groups includes identifying corresponding data entry values in the generated feature vectors of images of the reference group. Statistical values associated with the identified corresponding data entry values are calculated and then the calculated statistical values are combined to form the reference statistical vector.

At 206, an input image group (e.g., input image group 116) is received as input and input feature vectors (e.g., input feature vectors 118) are generated of the images of the input image group. It should be understood that the generation of the input feature vectors is performed in the same manner as the generation of the reference feature vectors. For instance, in an example, an input image group comprising fifty images results in the generation of fifty input feature vectors associated with the input image group.

At 208, an input statistical vector (e.g., input statistical vector 120) is generated for the input image group using the generated input feature vectors. It should be understood that the generation of the input statistical vector is performed in the same manner as the generation of the reference statistical vectors. For instance, in an example, the input image group comprising fifty images results in the generation of a single input statistical vector associated with the input image group, wherein the input statistical vector includes statistical data values that describe the input feature vectors associated with the input image group.

At 210, outlier analysis is performed using the input statistical vector and one or more of the reference statistical vectors. In some examples, the outlier analysis is performed using an outlier analysis engine 114 as described above with respect to FIG. 1. The outlier analysis compares the data of the input statistical vector to the data in the reference statistical vectors and determines how similar and/or different they are. The degree of difference is compared to a difference threshold and, if the degree of difference exceeds the difference threshold, the input statistical vector is an outlier with respect to the analyzed reference statistical vectors. At 212, if the output of the outlier analysis indicates that the input statistical vector is an outlier, the process proceeds to 214. Alternatively, if the output of the outlier analysis indicates that the input statistical vector is not an outlier, the process proceeds to 216.

At 214, it is determined that the input image group is an outlier with respect to the analyzed reference image groups. In some examples, the determination of outlier status for the input image group results in the input image group being categorized and/or stored differently than the reference image groups. The outlier status of the input image group can be used for other purposes without departing from the description.

At 216, it is determined that the input image group is not an outlier with respect to the reference image group. In some examples, the determination of non-outlier status, or inlier status, for the input image group results in the input image being categorized and/or stored with the reference image groups. The inlier status of the input image group can be used for other purposes without departing from the description.

In some examples, the reference image groups are associated with a category of medical imaging and determining that the input image group is an outlier with respect to the reference image groups includes determining that the input image group is not associated with the category of medical imaging.

Further, in some examples, the reference image groups include subgroups associated with a first category and a second category. The outlier analysis is performed to compare the input statistical vector to reference statistical vectors associated with the subgroup of the first category and to compare the input statistical vector to reference statistical vectors associated with the subgroup of the second category. The outlier status of the input image group is determined with respect to the subgroup of the first category and the subgroup of the second category, such that the input image group can be categorized as part of the first category, part of the second category, and/or part of neither category. The input image group may then be labeled based on its status with respect to the first and/or second categories and/or stored or otherwise arranged in association with the first and/or second categories.

In some examples, the method 200 includes causing a data analysis action to be performed on a target plurality of image groups based, at least in part, on the outcome of the performed outlier analysis. If it is determined that the input image group is an outlier with respect to the reference image groups, the input image group is excluded from the target plurality of image groups and the data analysis action is caused to be performed on the target plurality of image groups. Alternatively, if it is determined that the input image group is an inlier with respect to the reference image groups, the data analysis action is caused to be performed on the input image group (e.g., as part of the target plurality of image groups). In some such examples, the data analysis action is associated with analysis of an image group category with which the reference image groups are associated (e.g., a specific type of medical imaging series or study, such as the studies of FIG. 6). Further, in some examples, the data analysis action includes automated analysis to identify common data patterns in the target plurality of image groups; automated analysis to generate graphs, charts, and/or other visualizations of statistical features of the target plurality of image groups; automated analysis to generate recommended user actions associated with the target plurality of image groups; and/or automated analysis to generate a training data set for use in training an image classification model.

It should be understood that, in other examples, the method 200 includes, in isolation or combination, other features and/or aspects described herein with respect to FIGS. 1, 3, 4, and/or 5 without departing from description.

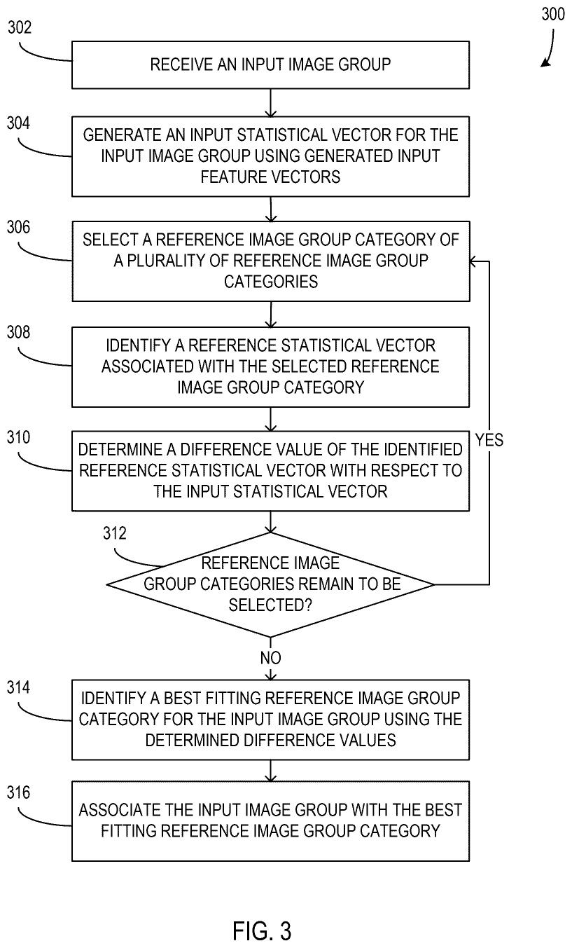

FIG. 3 is a flowchart illustrating an exemplary method 300 for categorizing an input image group using a plurality of reference image group categories. In some examples, the method 300 is executed or otherwise performed using a system such as system 100 of FIG. 1.

At 302, an input image group is received and, at 304, an input statistical vector for the input image group is generated using input feature vectors of the input image group. It should be understood that, in some examples, the generation of the input statistical vector is performed in the same manner as described above with respect to FIGS. 1 and 2.

At 306, a reference image group category is selected from a plurality of reference image group categories. In some examples, each reference image group category is associated with a plurality of reference image groups that share a category (e.g., a specific type of CT scan/study). Further, in some examples, the selection of the reference image group category includes determining which categories have not yet been selected during the method 300 and selecting a next category from that determined group of categories.

At 308, a reference statistical vector associated with the selected reference image group category is identified. In some examples, more than one reference statistical vector associated with the selected reference image group category is identified, rather than just a single reference statistical vector.

At 310, a difference value of the determined reference statistical vector with respect to the input statistical vector is determined. In some examples, the difference value is determined using outlier analysis as described herein. Further, in examples where multiple reference statistical vectors are identified at 308, the outlier analysis is applied to all of the identified reference statistical vectors with respect to the input statistical vector.

At 312, if reference image group categories remain to be selected, the process returns to 306. Alternatively, if no reference image group categories remain to be selected, the process proceeds to 314.

At 314, a best fitting reference image group category for the input image group is identified using the determined difference values. In some examples, the best fitting reference image group category is the category for which the smallest difference value was determined (e.g., the smallest difference exists between the input image group and the images of the reference image group category). In other examples, other methods of determining a best fitting reference image group category are used without departing from the description.

At 316, the input image group is associated with the best fitting reference image group category, such that the input image group becomes a part of the best fitting reference image group category. Additionally, or alternatively, in some examples, the generated input statistical vector is associated with the best fitting reference image group category so it can be used during future performances of the method 300 to categorize future input image groups.

It should be understood that, in other examples, the method 300 includes, in isolation or combination, other features and/or aspects described herein with respect to FIGS. 1, 2, 4, and/or 5 without departing from description.

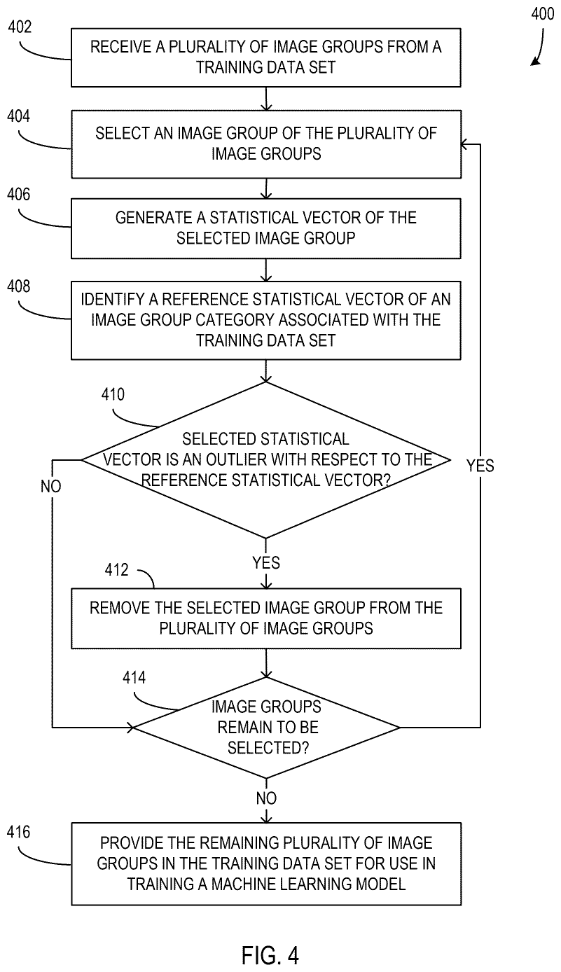

FIG. 4 is a flowchart illustrating an exemplary method 400 for filtering outlier image groups from a training data set of image groups. In some examples, the method 400 is executed or otherwise performed using a system such as system 100 of FIG. 1.

At 402, a training data set comprising a plurality of image groups is received. In some examples, the training data set is to be used to train a model to identify image groups associated with a specific category, but the training data set may contain outlier image groups that will inhibit the training process.

At 404, an image group of the plurality of image groups is selected. In some examples, selecting the image group includes determining which image groups remain to be selected and then selecting the next image group from that determined subset of the image groups.

At 406, a statistical vector of the selected image group is generated. In some examples, the generation of the statistical vector includes generating feature vectors for the images of the selected image group and then generating the statistical vector using those feature vectors, as described above with respect to FIG. 1.

At 408, a reference statistical vector of an image group category associated with the training data set is identified. In some examples, the image group category is the category for which the training data set will be used for training machine learning models. At 410, if the selected statistical vector is an outlier with respect to the reference statistical vector, the process proceeds to 412. Alternatively, if the selected statistical vector is not an outlier with respect to the reference statistical vector, the process proceeds to 414. In some examples, a plurality of reference statistical vectors associated with the image group category are identified and compared with the selected statistical vector to determine its outlier status.

At 412, the selected image group is removed from the plurality of image groups associated with the training data set and the process proceeds to 414.

At 414, if there are image groups of the plurality of image groups that remain to be selected, the process returns to 404. Alternatively, if there are no image groups of the plurality of image groups that remain to be selected, the process proceeds to 416.

At 416, the remaining plurality of image groups in the training data set are provided for use in training a machine learning model. As a result of the method 400, outlier image groups have been filtered out of the training data set such that it can be used to accurately train the machine learning model.

It should be understood that, in other examples, the method 400 includes, in isolation or combination, other features and/or aspects described herein with respect to FIGS. 1, 2, 3, and/or 5 without departing from description.

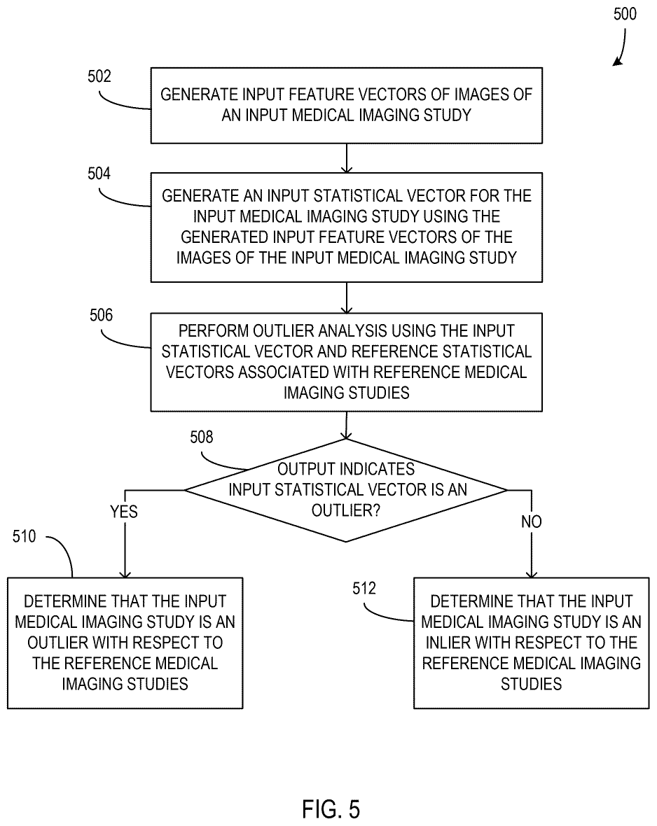

FIG. 5 is a flowchart illustrating an example method 500 for determining whether an input medical imaging study is an outlier with respect to a plurality of reference medical imaging studies. In some examples, the method 500 is executed or otherwise performed in a system, such as system 100 of FIG. 1, wherein the reference image groups 102 and input image group 116 are specifically medical imaging studies.

At 502, input feature vectors of images of an input medical imaging study are generated.

At 504, an input statistical vector is generated for the input medical imaging study using the generated input feature vectors of the images of the input medical imaging study.

At 506, outlier analysis is performed using the input statistical vector and reference statistical vectors associated with reference medical imaging studies.

At 508, it is determined whether the input medical imaging study is an outlier or an inlier with respect to the reference medical imaging studies based on the performed outlier analysis. If the input statistical vector is found to be an outlier, the process proceeds to 510. Alternatively, if the input statistical vector is found to be an inlier, the process proceeds to 512.

At 510, it is determined that the input medical imaging study is an outlier with respect to the reference medical imaging studies.

At 512, it is determined that the input medical imaging study is an inlier with respect to the reference medical imaging studies.

In some examples, the method 500 includes causing a data analysis action to be performed on a target plurality of medical imaging studies based, at least in part, on the outcome of the performed outlier analysis. If it is determined that the input medical imaging study is an outlier with respect to the reference medical imaging studies, the input medical imaging study is excluded from the target plurality of medical imaging studies and the data analysis action is caused to be performed on the target plurality of medical imaging studies. Alternatively, if it is determined that the input medical imaging study is an inlier with respect to the reference medical imaging studies, the data analysis action is caused to be performed on the input medical imaging study (e.g., as part of the target plurality of medical imaging studies). In some such examples, the data analysis action is associated with analysis of a medical imaging study category with which the reference medical imaging studies are associated (e.g., a specific type of medical imaging series or study). Further, in some examples, the data analysis action includes automated analysis to identify common data patterns in the target plurality of medical imaging studies; automated analysis to generate graphs, charts, and/or other visualizations of statistical features of the target plurality of medical imaging studies; automated analysis to generate recommended user actions associated with the target plurality of medical imaging studies; and/or automated analysis to generate a training data set for use in training an image classification model.

In some examples, the method 500 includes additional features for generating the reference statistical vectors associated with the reference medical imaging studies. For instance, in an example, generating a reference statistical vector of the reference statistical vectors associated with a reference medical imaging study of the reference medical imaging studies includes identifying corresponding data entry values in reference feature vectors of images of the reference medical imaging study, calculating statistical values associated with the identified corresponding data entry values, and combining the calculated statistical values to form the reference statistical vector.

In some examples, the method 500 includes additional features wherein the reference medical imaging studies include a first subgroup of the reference medical imaging studies associated with a first category and a second subgroup of the reference medical imaging studies associated with a second category; and wherein performing the outlier analysis using the input statistical vector and the reference statistical vectors includes performing outlier analysis using the input statistical vector and the reference statistical vectors associated with the first subgroup of the reference medical imaging studies associated with the first category, and performing outlier analysis using the input statistical vector and the reference statistical vectors associated with the second subgroup of the reference medical imaging studies associated with the second category. Further, determining that the input medical imaging study is an outlier with respect to the reference medical imaging studies based on the performed outlier analysis further includes determining that the input medical imaging study is an outlier with respect to the first subgroup of reference medical imaging studies associated with the first category and determining the input medical imaging study is an inlier with respect to the second subgroup of reference medical imaging studies associated with the second category.

In some examples including the features described in the immediately previous paragraph, the method 500 includes additional aspects wherein the input medical imaging study is labeled as being associated with the second category and not associated with the first category and wherein the input medical imaging study is added to the second subgroup of reference medical imaging studies associated with the second category.

In some examples, the method 500 includes additional features wherein reference feature vectors of a plurality of images in the reference medical imaging studies are generated, wherein generating the reference feature vectors includes providing the plurality of images to a trained vision model as input and receiving the generated reference feature vectors as output from the trained vision model.

In some examples, the method 500 includes additional features wherein excluding the input medical imaging study from a target plurality of medical imaging studies includes removing the input medical imaging study from a training data set used to train an image classification model and wherein causing a data analysis action to be performed on the target plurality of medical imaging studies includes training the image classification model using the training data set from which the input medical imaging study was removed.

In some examples, the method 500 includes additional features wherein the input medical imaging study includes a medical imaging series associated with at least one of X-ray imaging, computed tomography (CT) imaging, magnetic resonance imaging (MRI), Ultrasound imaging, and positron emission tomography (PET) imaging.

It should be understood that the additional features described above with respect to method 500 are not inextricably linked to the other features unless otherwise noted and, in other examples, the method 500 includes, in isolation or combination, other features and/or aspects described herein with respect to FIGS. 1, 2, 3, and/or 4 without departing from description.

FIG. 6 is a diagram 600 illustrating structures of two example medical imaging studies 602 and 616. In some examples, the medical imaging studies 602 and/or 616 are included in reference medical imaging studies as described above with respect to FIGS. 1-5 and/or included as input medical imaging studies as described above with respect to FIGS. 1-5.

Further, in some examples, medical imaging studies include one or more medical imaging series, which include one or more medical images. As illustrated, the CT study 602 includes an X-ray “scout” series 604, a CT volume series 606, and/or other series without departing from the description. The mammography study 616 includes an X-ray series 618 and an X-ray series 620, wherein each series is associated with scanning a specific portion of a patient's anatomy. Each medical imaging series includes one or more images. As illustrated, the X-ray “scout” series 604 includes a single X-ray image 608 (e.g., an image of the portion of anatomy to be CT scanned) and the CT volume series 606 includes a plurality of CT images 610 and 612-614. Further, the X-ray series 618 includes X-ray images 622 and 624-626 and the X-ray series 620 includes X-ray images 628 and 630-632.

More generally, a medical imaging series includes images associated with a type of scan or imaging process, such as X-rays, MRI, CT scans, Ultrasound, positron emission tomography (PET) scans, or the like. While each medical imaging series includes images from a single type of scan or imaging process, a medical imaging study can include medical imaging series from multiple types of scans or imaging processes as well as other types of data, as illustrated in the CT study 602, which includes an X-ray series and a CT series. In other examples, more, fewer, or different types of medical imaging series are included in the medical imaging studies without departing from the description. It should be understood that a first medical imaging study may have different series with different quantities of images than a second medical imaging study and the described systems and methods can be used to compare the first and second medical imaging studies as described herein. Further, the systems and methods can be used to compare medical image series to each other as well. In the case of the two illustrated studies 602 and 616, the outlier detection between the two studies 602 and 616 would be trivial due to the significance of the differences therebetween. The described systems and methods are configured to detect differences between studies even when the differences are more subtle.

### Additional Examples

In some examples, the described image group outlier identification and/or image group categorization methods are used to organize, categorize, filter, or otherwise automatically process groups of medical images with respect to clinical trials and/or associated medical research. Additionally, or alternatively, the described methods and systems have other data science applications as well.

In an example, the described methods are first used with a plurality of image groups in order to determine the image groups that fit into a specific medical image study category. Each image group is compared to data associated with reference image groups of the category and image groups that are found to be outliers are filtered out into a second plurality of image groups. Once that filtering step is complete, the image groups of the second plurality of image groups can be categorized further based on, for instance, reasons that the image groups were considered outliers. For instance, it may be determined that particular subset of the second plurality of image groups are outliers because they contain blurry images, while another subset of the second plurality of image groups are outliers because they cover incomplete anatomy. These additional image group categories can then be used during future analyses of input image groups to detect specific defects in those image groups. Other types of sub-categorization can be done without departing from the description.

### Exemplary Operating Environment

The present disclosure is operable with a computing apparatus according to an embodiment as a functional block diagram 700 in FIG. 7. In an example, components of a computing apparatus 718 are implemented as a part of an electronic device according to one or more embodiments described in this specification. The computing apparatus 718 comprises one or more processors 719 which may be microprocessors, controllers, or any other suitable type of processors for processing computer executable instructions to control the operation of the electronic device. Alternatively, or in addition, the processor 719 is any technology capable of executing logic or instructions, such as a hard-coded machine. In some examples, platform software comprising an operating system 720 or any other suitable platform software is provided on the apparatus 718 to enable application software 721 to be executed on the device. In some examples, identifying outlier image groups with respect to reference image groups as described herein is accomplished by software, hardware, and/or firmware.

In some examples, computer executable instructions are provided using any computer-readable media that is accessible by the computing apparatus 718. Computer-readable media include, for example, computer storage media such as a memory 722 and communications media. Computer storage media, such as a memory 722, include volatile and non-volatile, removable, and non-removable media implemented in any method or technology for storage of information such as computer readable instructions, data structures, program modules or the like. Computer storage media include, but are not limited to, Random Access Memory (RAM), Read-Only Memory (ROM), Erasable Programmable Read-Only Memory (EPROM), Electrically Erasable Programmable Read-Only Memory (EEPROM), persistent memory, phase change memory, flash memory or other memory technology, Compact Disk Read-Only Memory (CD-ROM), digital versatile disks (DVD) or other optical storage, magnetic cassettes, magnetic tape, magnetic disk storage, shingled disk storage or other magnetic storage devices, or any other non-transmission medium that can be used to store information for access by a computing apparatus. In contrast, communication media may embody computer readable instructions, data structures, program modules, or the like in a modulated data signal, such as a carrier wave, or other transport mechanism. As defined herein, computer storage media does not include communication media. Therefore, a computer storage medium is not a propagating signal. Propagated signals are not examples of computer storage media. Although the computer storage medium (the memory 722) is shown within the computing apparatus 718, it will be appreciated by a person skilled in the art, that, in some examples, the storage is distributed or located remotely and accessed via a network or other communication link (e.g., using a communication interface 723).

Further, in some examples, the computing apparatus 718 comprises an input/output controller 724 configured to output information to one or more output devices 725, for example a display or a speaker, which are separate from or integral to the electronic device. Additionally, or alternatively, the input/output controller 724 is configured to receive and process an input from one or more input devices 726, for example, a keyboard, a microphone, or a touchpad. In one example, the output device 725 also acts as the input device. An example of such a device is a touch sensitive display. The input/output controller 724 may also output data to devices other than the output device, e.g., a locally connected printing device. In some examples, a user provides input to the input device(s) 726 and/or receives output from the output device(s) 725.

The functionality described herein can be performed, at least in part, by one or more hardware logic components. According to an embodiment, the computing apparatus 718 is configured by the program code when executed by the processor 719 to execute the embodiments of the operations and functionality described. Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (ASICs), Program-specific Standard Products (ASSPs), System-on-a-chip systems (SOCs), Complex Programmable Logic Devices (CPLDs), Graphics Processing Units (GPUs).

At least a portion of the functionality of the various elements in the figures may be performed by other elements in the figures, or an entity (e.g., processor, web service, server, application program, computing device, or the like) not shown in the figures.

Although described in connection with an exemplary computing system environment, examples of the disclosure are capable of implementation with numerous other general purpose or special purpose computing system environments, configurations, or devices.

Examples of well-known computing systems, environments, and/or configurations that are suitable for use with aspects of the disclosure include, but are not limited to, mobile or portable computing devices (e.g., smartphones), personal computers, server computers, hand-held (e.g., tablet) or laptop devices, multiprocessor systems, gaming consoles or controllers, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, and the like. In general, the disclosure is operable with any device with processing capability such that it can execute instructions such as those described herein. Such systems or devices accept input from the user in any way, including from input devices such as a keyboard or pointing device, via gesture input, proximity input (such as by hovering), and/or via voice input.

Examples of the disclosure may be described in the general context of computer-executable instructions, such as program modules, executed by one or more computers or other devices in software, firmware, hardware, or a combination thereof. The computer-executable instructions may be organized into one or more computer-executable components or modules. Generally, program modules include, but are not limited to, routines, programs, objects, components, and data structures that perform particular tasks or implement particular abstract data types. Aspects of the disclosure may be implemented with any number and organization of such components or modules. For example, aspects of the disclosure are not limited to the specific computer-executable instructions, or the specific components or modules illustrated in the figures and described herein. Other examples of the disclosure include different computer-executable instructions or components having more or less functionality than illustrated and described herein.

In examples involving a general-purpose computer, aspects of the disclosure transform the general-purpose computer into a special-purpose computing device when configured to execute the instructions described herein.

An example system comprises a processor; and a memory comprising computer program code, the memory and the computer program code configured to cause the processor to: generate input feature vectors of images of an input medical imaging study; generate an input statistical vector for the input medical imaging study using the generated input feature vectors of the images of the input medical imaging study; perform outlier analysis using the input statistical vector and reference statistical vectors associated with reference medical imaging studies; determine that the input medical imaging study is an outlier with respect to the reference medical imaging studies based on the performed outlier analysis; exclude the input medical imaging study from a target plurality of medical imaging studies based on determining that the input medical imaging study is an outlier with respect to the reference medical imaging studies; and cause a data analysis action to be performed on the target plurality of medical imaging studies, wherein the data analysis action is associated with analysis of a medical imaging study category with which the reference medical imaging studies are associated.

An example computerized method comprises generating reference feature vectors of a plurality of images in reference image groups; generating reference statistical vectors for the reference image groups using the generated reference feature vectors of images included in the reference image groups, wherein a reference statistical vector is generated for each reference image group; generating input feature vectors of images of an input image group; generating an input statistical vector for the input image group using the generated input feature vectors of the images of the input image group; performing outlier analysis using the input statistical vector and the reference statistical vectors; determining that the input image group is an inlier with respect to the reference image groups based on the performed outlier analysis; and causing a data analysis action to be performed on the input image group based on determining that the input image group is an inlier with respect to the reference image groups, wherein the data analysis action is associated with analysis of an image group category with which the reference image groups are associated.

One or more computer storage media have computer-executable instructions that, upon execution by a processor, cause the processor to at least: generate input feature vectors of images of an input image group; generate an input statistical vector for the input image group using the generated input feature vectors of the images of the input image group; perform outlier analysis using the input statistical vector and reference statistical vectors associated with reference image groups; determine that the input image group is an outlier with respect to the reference image groups based on the performed outlier analysis; exclude the input image group from a target plurality of image groups based on determining that the input image group is an outlier with respect to the reference image groups; and cause a data analysis action to be performed on the target plurality of image groups, wherein the data analysis action is associated with analysis of an image group category with which the reference image groups are associated.

Alternatively, or in addition to the other examples described herein, examples include any combination of the following:
wherein the reference image groups are associated with a category of medical imaging and determining that the input image group is an outlier with respect to the reference image groups includes determining that the input image group is not associated with the category of medical imaging.
wherein generating the reference statistical vector for a reference image group of the reference image groups includes: identifying corresponding data entry values in the generated reference feature vectors of images of the reference image group; calculating statistical values associated with the identified corresponding data entry values; and combining the calculated statistical values to form the reference statistical vector.
wherein the reference image groups include a first subgroup of the reference image groups associated with a first category and a second subgroup of the reference image groups associated with a second category; and wherein performing the outlier analysis using the input statistical vector and the reference statistical vectors includes: performing outlier analysis using the input statistical vector and the reference statistical vectors associated with the first subgroup of the reference image groups associated with the first category; and performing outlier analysis using the input statistical vector and the reference statistical vectors associated with the second subgroup of the reference image groups associated with the second category; and wherein determining that the input image group is an outlier with respect to the reference image groups based on the performed outlier analysis further includes: determining that the input image group is an outlier with respect to the first subgroup of reference image groups associated with the first category; and determining the input image group is not an outlier with respect to the second subgroup of reference image groups associated with the second category.
wherein the input image group is labeled as being associated with the second category and not associated with the first category; and wherein the input image group is added to the second subgroup of reference image groups associated with the second category.
wherein generating reference feature vectors of a plurality of images in reference image groups includes providing the plurality of images to a trained vision model as input and receiving the generated reference feature vectors as output from the trained vision model.
further comprising: removing the input image group from a training data set used to train an image classification model; and training the image classification model using the training data set from which the input image group was removed.
wherein the memory and the computer program code are configured to further cause the processor to generate the reference statistical vectors associated with the reference medical imaging studies; wherein generating a reference statistical vector of the reference statistical vectors associated with a reference medical imaging study of the reference medical imaging studies includes: identifying corresponding data entry values in reference feature vectors of images of the reference medical imaging study; calculating statistical values associated with the identified corresponding data entry values; and combining the calculated statistical values to form the reference statistical vector. -wherein the reference medical imaging studies include a first subgroup of the reference medical imaging studies associated with a first category and a second subgroup of the reference medical imaging studies associated with a second category; and wherein performing the outlier analysis using the input statistical vector and the reference statistical vectors includes: performing outlier analysis using the input statistical vector and the reference statistical vectors associated with the first subgroup of the reference medical imaging studies associated with the first category; and performing outlier analysis using the input statistical vector and the reference statistical vectors associated with the second subgroup of the reference medical imaging studies associated with the second category; and wherein determining that the input medical imaging study is an outlier with respect to the reference medical imaging studies based on the performed outlier analysis further includes: determining that the input medical imaging study is an outlier with respect to the first subgroup of reference medical imaging studies associated with the first category; and determining the input medical imaging study is an inlier with respect to the second subgroup of reference medical imaging studies associated with the second category.
wherein the input medical imaging study is labeled as being associated with the second category and not associated with the first category; and wherein the input medical imaging study is added to the second subgroup of reference medical imaging studies associated with the second category.
wherein the memory and the computer program code are configured to further cause the processor to generate reference feature vectors of a plurality of images in the reference medical imaging studies, wherein generating the reference feature vectors includes providing the plurality of images to a trained vision model as input and receiving the generated reference feature vectors as output from the trained vision model.
wherein excluding the input medical imaging study from a target plurality of medical imaging studies includes removing the input medical imaging study from a training data set used to train an image classification model; and wherein causing a data analysis action to be performed on the target plurality of medical imaging studies includes training the image classification model using the training data set from which the input medical imaging study was removed.
wherein the input medical imaging study includes a medical imaging series associated with at least one of X-ray imaging, computed tomography (CT) imaging, magnetic resonance imaging (MRI), Ultrasound imaging, and positron emission tomography (PET) imaging.

Any range or device value given herein may be extended or altered without losing the effect sought, as will be apparent to the skilled person.

Examples have been described with reference to data monitored and/or collected from the users (e.g., user identity data with respect to profiles). In some examples, notice is provided to the users of the collection of the data (e.g., via a dialog box or preference setting) and users are given the opportunity to give or deny consent for the monitoring and/or collection. The consent takes the form of opt-in consent or opt-out consent.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

It will be understood that the benefits and advantages described above may relate to one embodiment or may relate to several embodiments. The embodiments are not limited to those that solve any or all of the stated problems or those that have any or all of the stated benefits and advantages. It will further be understood that reference to ‘an’ item refers to one or more of those items.

The embodiments illustrated and described herein as well as embodiments not specifically described herein but within the scope of aspects of the claims constitute an exemplary means for generating reference feature vectors of a plurality of images in reference image groups; exemplary means for generating reference statistical vectors for the reference image groups using the generated reference feature vectors of images included in the reference image groups, wherein a reference statistical vector is generated for each reference image group; exemplary means for generating input feature vectors of images of an input image group; exemplary means for generating an input statistical vector for the input image group using the generated input feature vectors of the images of the input image group; exemplary means for performing outlier analysis using the input statistical vector and the reference statistical vectors; exemplary means for determining that the input image group is an inlier with respect to the reference image groups based on the performed outlier analysis; and exemplary means for causing a data analysis action to be performed on the input image group based on determining that the input image group is an inlier with respect to the reference image groups, wherein the data analysis action is associated with analysis of an image group category with which the reference image groups are associated.

The term “comprising” is used in this specification to mean including the feature(s) or act(s) followed thereafter, without excluding the presence of one or more additional features or acts.

In some examples, the operations illustrated in the figures are implemented as software instructions encoded on a computer readable medium, in hardware programmed or designed to perform the operations, or both. For example, aspects of the disclosure are implemented as a system on a chip or other circuitry including a plurality of interconnected, electrically conductive elements.

The order of execution or performance of the operations in examples of the disclosure illustrated and described herein is not essential, unless otherwise specified. That is, the operations may be performed in any order, unless otherwise specified, and examples of the disclosure may include additional or fewer operations than those disclosed herein. For example, it is contemplated that executing or performing a particular operation before, contemporaneously with, or after another operation is within the scope of aspects of the disclosure.

When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The term “exemplary” is intended to mean “an example of.” The phrase “one or more of the following: A, B, and C” means “at least one of A and/or at least one of B and/or at least one of C.”

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

What is claimed is:

1. A system comprising:
a processor; and
a memory comprising computer program code, the memory and the computer program code configured to cause the processor to:
generate input feature vectors of images of an input medical imaging study;
generate an input statistical vector for the input medical imaging study using the generated input feature vectors of the images of the input medical imaging study;
perform outlier analysis using the input statistical vector and reference statistical vectors associated with reference medical imaging studies;
determine that the input medical imaging study is an outlier with respect to the reference medical imaging studies based on the performed outlier analysis;
exclude the input medical imaging study from a target plurality of medical imaging studies based on determining that the input medical imaging study is an outlier with respect to the reference medical imaging studies; and
cause a data analysis action to be performed on the target plurality of medical imaging studies, wherein the data analysis action is associated with analysis of a medical imaging study category with which the reference medical imaging studies are associated.

2. The system of claim 1, wherein the memory and the computer program code are configured to further cause the processor to generate the reference statistical vectors associated with the reference medical imaging studies;
wherein generating a reference statistical vector of the reference statistical vectors associated with a reference medical imaging study of the reference medical imaging studies includes:
identifying corresponding data entry values in reference feature vectors of images of the reference medical imaging study;
calculating statistical values associated with the identified corresponding data entry values; and
combining the calculated statistical values to form the reference statistical vector.

3. The system of claim 1, wherein the reference medical imaging studies include a first subgroup of the reference medical imaging studies associated with a first category and a second subgroup of the reference medical imaging studies associated with a second category; and
wherein performing the outlier analysis using the input statistical vector and the reference statistical vectors includes:
performing outlier analysis using the input statistical vector and the reference statistical vectors associated with the first subgroup of the reference medical imaging studies associated with the first category; and
performing outlier analysis using the input statistical vector and the reference statistical vectors associated with the second subgroup of the reference medical imaging studies associated with the second category; and
wherein determining that the input medical imaging study is an outlier with respect to the reference medical imaging studies based on the performed outlier analysis further includes:
determining that the input medical imaging study is an outlier with respect to the first subgroup of reference medical imaging studies associated with the first category; and
determining the input medical imaging study is an inlier with respect to the second subgroup of reference medical imaging studies associated with the second
category.

4. The system of claim 3, wherein the input medical imaging study is labeled as being associated with the second category and not associated with the first category; and
wherein the input medical imaging study is added to the second subgroup of reference medical imaging studies associated with the second category.

5. The system of claim 1, wherein the memory and the computer program code are configured to further cause the processor to generate reference feature vectors of a plurality of images in the reference medical imaging studies, wherein generating the reference feature vectors includes providing the plurality of images to a trained vision model as input and receiving the generated reference feature vectors as output from the trained vision model.

6. The system of claim 1, wherein excluding the input medical imaging study from a target plurality of medical imaging studies includes removing the input medical imaging study from a training data set used to train an image classification model; and
wherein causing a data analysis action to be performed on the target plurality of medical imaging studies includes training the image classification model using the training data set from which the input medical imaging study was removed.

7. The system of claim 1, wherein the input medical imaging study includes a medical imaging series associated with at least one of X-ray imaging, computed tomography (CT) imaging, magnetic resonance imaging (MRI), Ultrasound imaging, and positron emission tomography (PET) imaging.

8. A computerized method comprising:
generating reference feature vectors of a plurality of images in reference image groups;
generating reference statistical vectors for the reference image groups using the generated reference feature vectors of images included in the reference image groups, wherein a reference statistical vector is generated for each reference image group;
generating input feature vectors of images of an input image group;
generating an input statistical vector for the input image group using the generated input feature vectors of the images of the input image group;
performing outlier analysis using the input statistical vector and the reference statistical vectors;
determining that the input image group is an inlier with respect to the reference image groups based on the performed outlier analysis; and
causing a data analysis action to be performed on the input image group based on determining that the input image group is an inlier with respect to the reference image groups, wherein the data analysis action is associated with analysis of an image group category with which the reference image groups are associated.

9. The computerized method of claim 8, wherein the reference image groups are associated with a category of medical imaging and determining that the input image group is an inlier with respect to the reference image groups includes determining that the input image group is associated with the category of medical imaging.

10. The computerized method of claim 8, wherein generating the reference statistical vector for a reference image group of the reference image groups includes:
identifying corresponding data entry values in the generated reference feature vectors of images of the reference image group;
calculating statistical values associated with the identified corresponding data entry values; and
combining the calculated statistical values to form the reference statistical vector.

11. The computerized method of claim 8, wherein the reference image groups include a first subgroup of the reference image groups associated with a first category and a second subgroup of the reference image groups associated with a second category; and
wherein performing the outlier analysis using the input statistical vector and the reference statistical vectors includes:
performing outlier analysis using the input statistical vector and the reference statistical vectors associated with the first subgroup of the reference image groups associated with the first category; and
performing outlier analysis using the input statistical vector and the reference statistical vectors associated with the second subgroup of the reference image groups associated with the second category; and
wherein determining that the input image group is an inlier with respect to the reference image groups based on the performed outlier analysis further includes:
determining that the input image group is an outlier with respect to the first subgroup of reference image groups associated with the first category; and
determining the input image group is an inlier with respect to the second subgroup of reference image groups associated with the second category.

12. The computerized method of claim 11, wherein the input image group is labeled as being associated with the second category and not associated with the first category; and
wherein the input image group is added to the second subgroup of reference image groups associated with the second category.

13. The computerized method of claim 8, wherein generating reference feature vectors of a plurality of images in reference image groups includes providing the plurality of images to a trained vision model as input and receiving the generated reference feature vectors as output from the trained vision model, wherein the trained vision model is not trained specifically for use with the reference image groups.

14. The computerized method of claim 8, further comprising:
removing the input image group from a training data set used to train an image classification model; and
training the image classification model using the training data set from which the input image group was removed.

15. A computer storage medium has computer-executable instructions that, upon execution by a processor, cause the processor to at least:
generate input feature vectors of images of an input image group;
generate an input statistical vector for the input image group using the generated input feature vectors of the images of the input image group;
perform outlier analysis using the input statistical vector and reference statistical vectors associated with reference image groups;
determine that the input image group is an outlier with respect to the reference image groups based on the performed outlier analysis;
exclude the input image group from a target plurality of image groups based on determining that the input image group is an outlier with respect to the reference image groups; and
cause a data analysis action to be performed on the target plurality of image groups, wherein the data analysis action is associated with analysis of an image group category with which the reference image groups are associated.

16. The computer storage medium of claim 15, wherein the reference image groups are associated with a category of medical imaging and determining that the input image group is an outlier with respect to the reference image groups includes determining that the input image group is not associated with the category of medical imaging.

17. The computer storage medium of claim 15, wherein generating the input statistical vector for the input image group includes:
identifying corresponding data entry values in the generated input feature vectors of images of the input image group;
calculating statistical values associated with the identified corresponding data entry values; and
combining the calculated statistical values to form the input statistical vector.

18. The computer storage medium of claim 15, wherein the reference image groups include a first subgroup of the reference image groups associated with a first category and a second subgroup of the reference image groups associated with a second category; and
wherein performing the outlier analysis using the input statistical vector and the reference statistical vectors includes:
performing outlier analysis using the input statistical vector and the reference statistical vectors associated with the first subgroup of the reference image groups associated with the first category; and
performing outlier analysis using the input statistical vector and the reference statistical vectors associated with the second subgroup of the reference image groups associated with the second category; and
wherein determining that the input image group is an outlier with respect to the reference image groups based on the performed outlier analysis further includes:
determining that the input image group is an outlier with respect to the first subgroup of reference image groups associated with the first category; and
determining the input image group is an inlier with respect to the second subgroup of reference image groups associated with the second category.

19. The computer storage medium of claim 18, wherein the input image group is labeled as being associated with the second category and not associated with the first category; and
wherein the input image group is added to the second subgroup of reference image groups associated with the second category.

20. The computer storage medium of claim 15, wherein the computer-executable instructions, upon execution by a processor, further cause the processor to at least:
remove the input image group from a training data set used to train an image classification model; and
train the image classification model using the training data set from which the input image group was removed.
